(19) United States
(12) Patent Application Publication
(10) Pub. No.: US 2025/0222134 A1
(43) Pub. Date: Jul. 10, 2025

(54) COMPOSITIONS AND METHODS FOR THE TREATMENT OF GLYCOGEN STORAGE DISORDERS

(71) Applicant: University of Connecticut, Farmington, CT (US)

(72) Inventors: Youngmok Lee, Farmington, CT (US); Junho Cho, Rockville, MD (US)

(21) Appl. No.: 18/630,651

(22) PCT Filed: Oct. 17, 2022
(86) PCT No.: PCT/US2022/078199

§ 371 Date: Apr. 9, 2024

Related U.S. Application Data
(60) Provisional application No. 63/257,191, filed on Oct. 19, 2021.

(51) Int. Cl. A61K 48/00; A61K 9/00; A61K 38/44; A61P 3/00; A61P 37/06; C12N 15/86

(52) U.S. Cl. CPC A61K 48/005; A61K 9/0019; A61K 38/443; A61K 48/0075; A61K 48/0083; A61P 3/00; A61P 37/06; C12N 15/86; C12Y 101/01049; C12N 2740/15043; C12N 2750/14143; C12N 2800/22; C12N 2830/48; C12N 2830/50

(57) ABSTRACT

Described herein are gene therapy treatments for glycogen storage disorders, the treatments comprising AAV vectors shown to express human glucose-6-phosphate transporter gene in the liver, kidney, and immune cells of a subject.

### CROSS-REFERENCE TO RELATED APPLICATIONS

This application claims priority to U.S. Provisional Application 63/247,191 filed on Oct. 19, 2021, which is incorporated herein by reference in its entirety.

### SEQUENCE LISTING

The Instant Application contains a Sequence Listing which has been submitted electronically in ASCII format and is hereby incorporated by reference in its entirety. Said ASCII copy, created on Oct. 10, 2022 is named “UCT0300PCT” and is 22,528 bytes in size.

### BACKGROUND

Glycogen storage disease type Ib (GSD-Ib, MIM232220) is caused by a deficiency in the ubiquitously expressed glucose-6-phosphate (G6P) transporter (G6PT or SLC37A4), which translocates G6P from the cytoplasm into the lumen of the endoplasmic reticulum (ER). Inside the ER, G6P is hydrolyzed to glucose and phosphate by either the liver/kidney/intestine-restricted glucose-6-phosphatase-α (G6Pase-α or G6PC) or the ubiquitously expressed G6Pase-β. G6PT and G6Pase are functionally co-dependent and form the G6PT/G6Pase complexes. The G6PT/G6Pase-α complex maintains interprandial blood glucose homeostasis. A deficiency of either protein results in an abnormal metabolic phenotype characterized by fasting hypoglycemia, hepatocellular adenoma (HCA) and hepatocellular carcinoma (HCC), nephromegaly, hyperlipidemia, hyperuricemia, lactic acidemia, and growth retardation. The G6PT/G6Pase-β complex maintains neutrophil/macrophage homeostasis and function, and a deficiency of either protein results in neutropenia and myeloid dysfunction. Therefore GSD-Ib is not only a metabolic but also an immune disorder characterized by impaired glucose homeostasis, neutropenia, and myeloid dysfunction. Untreated GSD-Ib is juvenile lethal. Strict compliance with dietary therapies have enabled GSD-Ib patients to attain near normal growth and pubertal development.

Although precisely controlled uncooked cornstarch therapy is able to manage hypoglycemia and the related metabolic abnormalities, GSD-Ib patients are still exposed to the risk of hypoglycemic seizure or death by either a missing or uncontrolled dose of uncooked cornstarch.

Current therapies do not address some of the long-term effects of GSDIb. In addition, there is no cure for GSDIb. Accordingly, there is need in the art for improved therapies to more effectively treat GSDIb.

### SUMMARY

Disclosed herein is a recombinant nucleic acid molecule and a recombinant viral vector, for example an adenovirus vector (rAAV), for use in a method of gene therapy for prevention and treatment GSD-Ib in a subject. The method comprises administering an effective dose or multiple doses of the rAAV. The method has been shown to prevent hypoglycemic seizure and normalizes metabolic disturbance in blood of a subject with a glycogen disease disorder, thereby reducing the chance of a life-threatening event of hypoglycemia in the subject, and prevent HCA and HCC.

The above described and other features are exemplified by the following figures and detailed description.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-D show evaluation of promoters. FIG. 1A shows the design of a candidate GFP expression vector driven by promoters from 5′ flanking sequences of human SLC37A4 gene. The nucleotide sequence of the promoters are identified in the Sequence Listing as shown below. FIGS. 1B-D show the promoter test with GFP expression strength revealed that 1 kb nucleotides of the 5′-flanking region of the human SLC37A4 (G6PT) gene induced the highest GFP expression levels between HepG2 (FIG. 1B), HEK293 (FIG. 1C), and HL60 cell line (FIG. 1D).

FIGS. 2A-B show codon optimization of human G6PT achieved significantly higher protein expression levels in human liver cell line (FIG. 2A, HepG2, 3.3 fold) and human kidney cell line (FIG. 2B, HEK293, 9.5 fold).

FIGS. 3A-B show survival rates of pAAV-GPT-co-opt-hG6PT treated G6pt−/− mice. Survival rate of newborn (NB) single-dose infused G6pt−/− mice and control mice (FIG. 3A) and Two-dose (NB and 12 weeks old) infused G6pt−/− mice and control mice (FIG. 3B) are shown.

FIGS. 4A-B show a fasting glucose test through 24 weeks old. Fasting glucose levels at 4 and 12 weeks after 1st dose are displayed in FIG. 4A. FIG. 4B shows a fasting glucose test after 2nd dose was administered.

FIG. 5 shows growth curves. GSD-Ib mice received AAV8-GTP-co-opt-h6PT vector neonatally (2-3×10¹³ vg/kg). The gene therapy enabled GSD-Ib mice survival, growth until normal size.

FIG. 6 shows sustained fasting glucose levels and blood metabolite levels through 24 weeks of age and corrected metabolite abnormalities at ages 4 weeks and 12 weeks in GSD-Ib mice receiving AAV8-GTP-co-opt-h6PT vector neonatally (2-3×10¹³ vg/kg).

FIG. 7 shows body weight, liver weight, liver weight/body weight percentage, and kidney weight/body weight percentage.

FIG. 8 reflects biodistribution analysis showing liver directed gene delivery.

FIG. 9 shows confirmation of mRNA expression and protein expression.

FIG. 10 shows absolute neutrophil counts in blood.

FIG. 11 shows a schematic of the gene therapy vector pAAV-GTP-Co-opt-hG6PT.

### SEQUENCE LISTING

In the nucleic and amino acid sequences listed in the accompanying sequence listing only one strand of each nucleic acid sequence is shown, but the complementary strand is understood as included by any reference to the displayed strand. In the accompanying sequence listing:

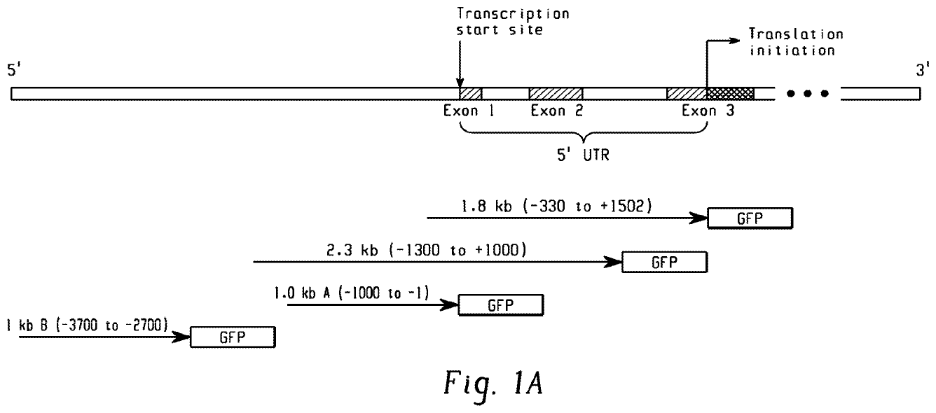

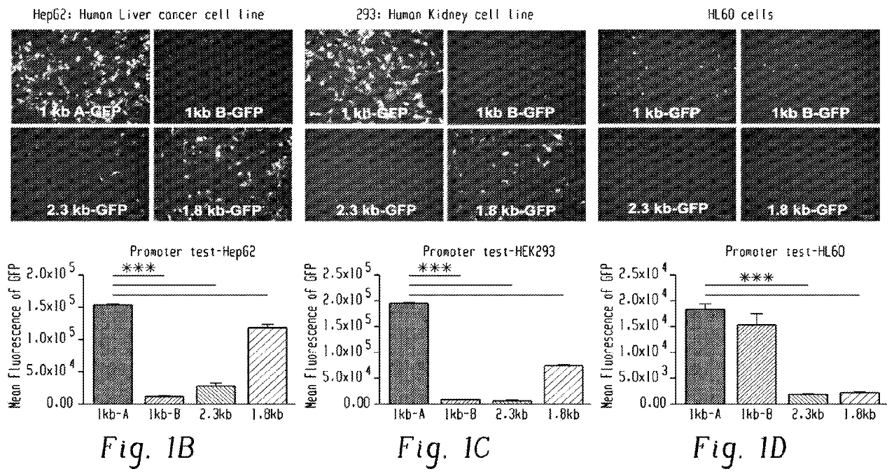

SEQ ID NO: 1 is the nucleotide sequence of pAAV-GTP-Co-opt-hG6PT having the following features:
5′ ITR: AAV 5′ inverted terminal repeat, nucleotides 1 to 141 (141 bp) of SEQ ID NO: 1
Native human G6PT promoter 1Kb A: Human G6PT gene 5′ Flanking Sequence −1000 to −1 upstream of the +1 transcription start site (FIG. 1A), nucleotides 169 to 1168 (1000 bp) of SEQ ID NO:1
Codon optimized human G6PT variant 1 coding sequence, nucleotides 1199 to 2488 (1290 bp) of SEQ ID NO:1
WPRE—Woodchuck hepatitis virus posttrancriptional regulatory element, nucleotides 2519-3116 (598 bp) of SEQ ID NO:1
BGH pA-Bovine growth hormone polyadenylation signal, nucleotides 3147-3354 (208 bp) of SEQ ID NO:1
3′ ITR, nucleotides 3362 to 3502 (141 bp) of SEQ ID NO:1.

SEQ ID NO: 2 is the promoter sequence 1 kb B shown in FIG. 1A, Human G6PT gene 5′ Flanking Sequence −3700 to −2700 upstream of the +1 transcription start site (FIG. 1A).

SEQ ID NO: 3 is the promoter sequence 1.8 kb shown in FIG. 1A, 1.8 kb of human G6PT gene sequence upstream of the translation initiation site, extending from −330 of the 5′ flanking region upstream of the +1 transcription start site to +1502 upstream of the translation initiation site (FIG. 1A).

SEQ ID NO: 4 is the promoter sequence 2.3 kb shown in FIG. 1A, 2.3 kb of human G6PT gene sequence upstream of the translation initiation site, extending from −1300 of the 5′ flanking region upstream of the +1 transcription initiation site to +1000 upstream of the translation initiation site (FIG. 1A).

SEQ ID NO: 5 is the native human G6PT coding sequence.

SEQ ID NO: 6 is the amino acid sequence of human G6PT.

SEQ ID NO:7 is the forward primer: gtgatcttca gcgccatgtt.

SEQ ID NO: 8 is the reverse primer: gaacttgctg atggcgtagg.

### DETAILED DESCRIPTION

### Definitions

Unless otherwise noted, technical terms are used according to conventional usage.

In order to facilitate review of the various embodiments of the disclosure, the following explanations of specific terms are provided:

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, intravenous, or renal vein injection), oral, intraductal, sublingual, rectal, transdermal, intranasal, vaginal and inhalation routes.

Enhancer: A nucleic acid sequence that increases the rate of transcription by increasing the activity of a promoter.

Glucose-6-phosphate transporter (G6PT): A gene located on human chromosome 11q23.3. The G6PT gene encodes a protein that regulates glucose-6-phosphate transport from the cytoplasm to the lumen of the ER in order to maintain glucose homeostasis. Mutations in the G6PT gene are associated with glycogen storage disease type Ib. G6PT is also known as solute carrier family 37 member 4 (SLC37A4).

Glycogen storage disease (GSD): A group of diseases that result from defects in the processing of glycogen synthesis or breakdown within muscles, liver and other tissues. GSD can either be genetic or acquired. Genetic GSD is caused by any inborn error of metabolism involved in these processes. There are currently 11 recognized glycogen storage diseases (GSD type I, II, III, IV, V, VI, VII, IX, XI, XII and XIII). GSD-I consists of two autosomal recessive disorders, GSD-Ia and GSD-Ib. GSD-Ia results from a deficiency in glucose-6-phosphatase-alpha. Deficiencies in the glucose-6-phosphate transporter (G6PT) are responsible for GSD-Ib.

Glycogen storage disease type Ib (GSD-Ib): An autosomal recessive disorder caused by deficiencies in glucose-6-phosphate transporter (G6PT), a ubiquitously expressed endoplasmic reticulum (ER) protein that translocate G6P from the cytoplasm into the ER lumen. GSD-Ib is both a metabolic disorder and an immune disorder. GSD-Ib metabolic abnormalities include fasting hypoglycemia, hepatomegaly, nephromegaly, hyperlipidemia, hyperuricemia, lactic acidemia and growth retardation. Although dietary therapies for GSD-Ib that significantly alleviate the metabolic abnormalities of GSD-Ib are available, patients continue to suffer from long-term complications of GSD-Ib, such as hepatocellular adenoma/carcinoma and renal disease. The GSD-Ib immunological abnormalities include neutropenia and myeloid dysfunction. Neutrophils from GSD-Ib patients exhibit impairment of chemotaxis, calcium mobilization, respiratory burst, and phagocytotic activities. As a result, recurrent bacterial infections are commonly seen and up to 77% of patients manifesting neutropenia also develop inflammatory bowel disease (IBD), indistinguishable from idiopathic Crohn's disease. As used herein, “treating GSD-Ib” refers to a therapeutic intervention that ameliorates one or more signs or symptoms of GSD-Ib or a pathological condition associated with GSD-Ib. Thus, “treating GSD-Ib” can include treating any metabolic or immune dysfunction associated with GSD-Ib, such as, but not limited to, hypoglycemia, hepatomegaly, nephromegaly, hyperlipidemia, hyperuricemia. lactic academia, growth retardation, neutropenia. myeloid dysfunction and IBD.

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Lentivirus: A genus of retroviruses characterized by a long incubation period and the ability to infect non-dividing cells. Lentiviruses are attractive gene therapy vectors due to their ability to provide long-term, stable gene expression and infect non-dividing cells. Examples of lentiviruses include human immunodeficiency virus (HIV), simian immunodeficiency virus (SIV). feline immunodeficiency virus (FIV), bovine immunodeficiency virus (BIV), caprine arthritis-encephalitis virus (CAEV) and equine infectious anemia virus (EIAV).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carrier: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill. tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Promoter: A region of DNA that directs/initiates transcription of a nucleic acid (e.g., a gene). A promoter includes necessary nucleic acid sequences near the start site of transcription. Typically, promoters are located near the genes they transcribe. A promoter also optionally includes distal enhancer or repressor elements which can be located as much as several thousand base pairs from the start site of transcription.

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

Similarly, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein. “recombinant AAV” refers to an AAV particle in which a recombinant nucleic acid molecule (such as a recombinant nucleic acid molecule encoding G6PT) has been packaged.

Sequence identity: The identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art.

The NCBI Basic Local Alignment Search Tool (BLAST) (is available from several sources, including the National Center for Biological Information (NCBI) and on the internet, for use in connection with the sequence analysis programs Blastp™, Blastn™ Blastx™, Tblastn™ and Tblastx™. Additional information can be found at the NCBI web site.

Serotype: A group of closely related microorganisms (such as viruses) distinguished by a characteristic set of antigens.

Subject: Living multi-cellular vertebrate organisms, a category that includes human and non-human mammals.

Synthetic: Produced by artificial means in a laboratory, for example a synthetic nucleic acid can be chemically synthesized in a laboratory.

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

“Treat” or “treating,” means to administer a therapeutic composition or agent of the disclosure or a product of the disclosure to a subject or patient having one or more disease symptoms, or being suspected of having a disease (such as GSD-Ib), for which the agent or product has therapeutic activity or prophylactic activity. The agent or product can be administered in an amount effective to alleviate one or more disease symptoms in the treated subject, whether by inducing the regression of or inhibiting the progression of such symptom(s) by any clinically measurable degree. The terms further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms.

“Preventing” means administering an amount of a pharmaceutical formulation of the disclosure or an agent of the disclosure or a product of the disclosure which is sufficient to significantly reduce the likelihood of a disease from occurring in a subject who may be predisposed to or have enhanced risk of getting the disease but who does not have it.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is a lentivirus vector or an AAV vector.

### DESCRIPTION

GSD-Ib (G6pt−/−) mice manifest both the metabolic and myeloid dysfunctions characteristic of human GSD-Ib. When left untreated, the G6pt−/− mice rarely survive weaning, reflecting the juvenile lethality seen in human patients. Previous studies have shown that systemic administration of a pseudotyped AAV2/8 vector expressing human G6PT directed by the chicken β-actin (CBA) promoter/CMV enhancer, delivers the G6PT transgene primarily to the liver. In doing so, it normalizes metabolic abnormalities in murine GSD-Ib. However, of the five treated G6pt−/− mice that survived for 51-72 weeks, two (40%) developed multiple HCAs with one undergoing malignant transformation.

Studies have shown that the choice of transgene promoter can impact targeting efficiency, tissue-specific expression, and the level of immune response or tolerance to the therapy. Indeed, for the related disease GSD-Ia, caused by a deficiency in G6Pase-α enzyme activity, a G6Pase-α-expressing rAAV vector directed by the native 2.8-kb human G6PC promoter/enhancer (GPE) provides sustained correction of metabolic abnormalities in murine GSD-Ia with no evidence of HCA. Moreover, the gluconeogenic tissue-specific GPE does not elicit the humoral response that was observed for the CBA promoter/CMV enhancer.

The vectors disclosed herein use either the GPE or a G6PT 5′-flanking region of the human SLC37A4 (G6PT) gene, consisting of nucleotides −1000 to −1 upstream of the +1 transcription start site of G6PT. The studies described herein examined the safety and efficacy of gene therapy in G6pt−/− mice using rAAV-GTP-co-opt-hG6PT vectors, which are rAAV8, rAAV9, or rAAV-quadYF vectors directed by the human G6PT promoter, respectively. Neonatal infusion of rAAV8-GTP-co-opt-hG6PT corrected metabolic abnormalities until adult age with minimal vector dilution based on fasting glucose levels. A second dose with two serotypes of AAV vectors (AAV9 and AAV-quadYF) influenced liver and kidney differently. Gene therapy in neonatal GSD-Ib mice enabled their survival, growth until normal size, ability to tolerate prolonged fasting, and corrected metabolic abnormalities at 4 weeks and 12 weeks of age.

Described herein are recombinant nucleic acid molecules, recombinant vectors, such as AAV and lentivirus vectors, and recombinant viruses, such as recombinant AAV and recombinant lentivirus, that can be used in gene therapy applications for the treatment of glycogen storage disease, specifically GSD-Ib.

Provided herein are recombinant nucleic acid molecules that include a human glucose-6-phosphate transporter (G6PT) codon optimized coding sequence operably linked to a human G6PT −1000 to −1 5′-flanking sequence (GPT). In some embodiments, the human G6PT promoter sequence consists of nucleotides 169-1168 of SEQ ID NO:1, or is at least 80%, at least 85%, at least 90%, at least 95%, at least 96%, at least 97%, at least 98% or at least 99% identical to nucleotides 169-1168 of SEQ ID NO: 1. In some aspects, the coding sequence of the codon optimized human G6PT gene is at least 80%, at least 85%, at least 90%, at least 95%, at least 96%, at least 97%, at least 98% or at least 99% identical to nucleotides 1199 to 2488 of SEQ ID NO: 1. In some examples, the human G6PT coding sequence comprises or consists of nucleotides 1199 to 2488 of SEQ ID NO: 1. In some embodiments, the codon optimized sequence includes the GPT promoter 1 kb-A (−1000 to −1 of 5′ flanking sequence of human G6PT gene) and is at least 80%, at least 85%, at least 90%, at least 95%, at least 96%, at least 97%, at least 98% or at least 99% identical to nucleotides 169 to 2488 of SEQ ID NO: 1 or is identical to nucleotides 169-2488 of SEQ ID NO:1. In some aspect, the GPT sequence comprises or consists of other regulatory elements such as Woodchuck hepatitis virus posttranscriptional regulatory element, a BGH polyA signal, and at least 80%, at least 85%, at least 90%, at least 95%, at least 96%, at least 97%, at least 98% or at least 99% identical to nucleotides 169 to 3354 of SEQ ID NO: 1, or is identical to nucleotides 169 to 3354 of SEQ ID NO: 1. In specific examples, the recombinant nucleic acid molecule comprises or consists of AAV 5′ inverted terminal repeat (ITR), the GPT promoter 1 kb-A, the codon optimized hG6PT gene, the woodchuck hepatitis virus posttransciptional regulatory element, the BGH polyA signal, and a 3′ITR, or the nucleotides 1 to 3502 of SEQ ID NO: 1 or a recombinant nucleic acid molecule that is at least 80%, at least 85%, at least 90%, at least 95%, at least 96%, at least 97%, at least 98% or at least 99% identical to nucleotides 1 to 3502 of SEQ ID NO: 1. In specific non-limiting examples, the recombinant nucleic acid molecule comprises or consists of the sequence identified in SEQ ID NO: 1.

Further provided are vectors comprising the recombinant nucleic acid molecules disclosed herein. In some embodiments, the vector is an AAV vector. The AAV serotype can be any suitable serotype for delivery of transgenes to a subject. In some examples, the AAV vector is a serotype 8 AAV (AAV8). In other examples the AAV vector is a serotype 1, 2, 3, 4, 5, 6, 7, 9, 10, 11 or 12 vector (i.e., AAV1, AAV2, AAV3, AAV4, AAV5, AAV6, AAV7, AAV9, AAV10, AAV11 or AAV12). In yet other examples, the AAV vector is a hybrid of two or more AAV serotypes (such as, but not limited to AAV2/1, AAV2/7, AAV2/8 or AAV2/9). In some aspect, the AAV vector is a QuadYF vector, an rAAV2-based capsid mutant vector (Y272F, Y444F, Y500F, Y730F, T491V; termed QuadYF+TV) with strong endothelial cell tropism at transducing the vasculature after systemic administration. The selection of AAV serotype will depend in part on the cell type(s) that are targeted for gene therapy. For treatment of GSD-Ib, the liver and kidney are the primary target organs. In other embodiments, the vector is a lentivirus vector. In some examples, the lentivirus vectors is an HIV, SIV, FIV, BIV, CAEV or EIAV vector.

Also provided herein are isolated host cells comprising the recombinant nucleic acid molecules or vectors disclosed herein. For example, the isolated host cell can be a cell (or cell line) appropriate for production of recombinant AAV (rAAV) or recombinant lentivirus. In some examples, the host cell is a mammalian cell, such as a HEK-293, HEK293T, HepG2, HL60, BHK, Vero, RD, HT-1080, A549, COS-1, Cos-7, ARPE-19, or MRC-5 cell.

Further provided are rAAV comprising a recombinant nucleic acid molecule disclosed herein. In some embodiments, the rAAV is rAAV8, rAAV9, and/or rAAV2 or rAAV2-QuadYF. However, the AAV serotype can be any other suitable AAV serotype, such as AAV1, AAV2, AAV3, AAV4, AAV5. AAV6, AAV7, AAV9, AAV10, AAV11 or AAV12, or a hybrid of two or more AAV serotypes (such as, but not limited to AAV2/1, AAV2/7, AAV2/8 or AAV2/9). Compositions comprising a rAAV disclosed herein and a pharmaceutically acceptable carrier are also provided by the present disclosure. In some embodiments, the compositions are formulated for intravenous or intramuscular administration. Suitable pharmaceutical formulations for administration of rAAV can be found, for example, in U.S. Patent Application Publication No. 2012/0219528. which is herein incorporated by reference.

Also provided are recombinant lentiviruses comprising a recombinant nucleic acid molecule disclosed herein. In some embodiments, the lentivirus is HIV, SIV, FIV, BIV, CAEV or EIAV. In particular examples, the lentivirus is HIV-1. Compositions comprising a recombinant lentivirus disclosed herein and a pharmaceutically acceptable carrier are also provided by the present disclosure. In some embodiments, the compositions are formulated for intravenous or intramuscular administration. In other embodiments, the recombinant lentivirus is formulated for ex vivo administration, such as for ex vivo administration to bone marrow cells.

Further provided are methods of treating a subject diagnosed with a glycogen storage disease, comprising selecting a subject with GSD-Ib and administering to the subject a therapeutically effective amount of a rAAV or recombinant lentivirus (or a composition comprising a rAAV or recombinant lentivirus) disclosed herein. In some embodiments, the rAAV or recombinant lentivirus is administered intravenously. In other embodiments, the recombinant virus is administered by retrograde renal vein injection.

In some embodiments, the subject to be treated exhibits one or more metabolic abnormalities associated with GSD-Ib. In some examples, the subject suffers from fasting hypoglycemia, hepatomegaly, nephromegaly, hyperlipidemia. hyperuricemia, lactic acidemia, and/or growth retardation. In some embodiments, the subject to be treated exhibits one or more immunological abnormalities associated with GSD-Ib. In some examples, the subject exhibits neutropenia, myeloid dysfunction, recurrent bacterial infection and/or inflammatory bowel disease (IBD).

In some embodiments, the rAAV is administered at a dose of about 1×10¹¹ to about 1×10¹⁴ viral genome (vg)/kg. In some examples, the rAAV is administered at a dose of about 1×10¹² to about 1×10¹⁴ vg/kg. In other examples, the rAAV is administered at a dose of about 5×10¹² to about 5×10¹³ vg/kg. In specific non-limiting examples, the rAAV is administered at a dose of at least about 1×10¹¹, at least about 5×10¹¹, at least about 1×10¹², at least about 51×10¹², at least about 1×10¹³, at least about 5×10¹³, or at least about 1×10¹⁴ vp/kg. In other non-limiting examples, the rAAV is administered at a dose of no more than about 5×10¹², no more than about 1×10¹². no more than about 5×10¹², no more than about 1×10¹³, no more than about 5×10¹³, or no more than about 1×10¹⁴ vg/kg. In specific non-limiting example, the rAAV is administered at a dose of about 0.7×10¹³ vg/kg, 2×10¹³ vg/kg, 1.4×10¹³ vg/kg or 4×10¹⁴ vg/kg. The rAAV can be administered in a single dose. or in multiple doses (such as 2, 3, 4, 5, 6, 7, 8, 9 or 10 doses) as needed for the desired therapeutic results.

### Recombinant AAV for Gene Therapy Applications

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21 (4): 583-593, 2008).

The left ORF of AAV contains the Rep gene, which encodes four proteins—Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging.

Methods for producing rAAV suitable for gene therapy are well known in the art, and can be utilized with the recombinant nucleic acid molecules and methods disclosed herein.

In some embodiments, the rAAV is provided as a lyophilized preparation and diluted in a virion-stabilizing composition for immediate or future use. Alternatively, the rAAV is provided immediately after production.

In some embodiments, the rAAV compositions contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally. auxiliary substances, such as wetting or emulsifying agents, pH buffering substances, and the like, may be present in such vehicles. Generally, excipients confer a protective effect on rAAV virions to minimize loss of rAAV, such as from formulation procedures, packaging, storage and transport. Excipients that are used to protect rAAV particles from degradative conditions include, but are not limited to, detergents, proteins. e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400. PEG-600, PEG-1000, PEG-1450, PEG-3350. PEG-6000, PEG-8000 and any molecular weights in between these values, propylene glycols (PG), sugar alcohols, such as a carbohydrate, for example sorbitol. The detergent, when present, can be an anionic, a cationic, a zwitterionic or a nonionic detergent. In some embodiments, the detergent is a nonionic detergent. In some examples, the nonionic detergent is a sorbitan ester, for example, polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85). In specific examples, the detergent is TWEEN®-20 and/or TWEEN®-80.

### Lentivirus Vectors for Gene Therapy Applications

Lentiviruses are a genus of retroviruses characterized by a long incubation period and the ability to infect non-dividing cells. Lentiviruses are complex retroviruses, which, in addition to the common retroviral genes gag, pol, and env, contain other genes with regulatory or structural function. The higher complexity enables the virus to modulate its life cycle, as in the course of latent infection. Examples of lentiviruses include HIV, SIV, FIV, SIV, BIV, CAEV and EIAV.

Lentiviral vectors have been generated by multiply attenuating the HIV virulence genes, for example, the genes env, vif, vpr, vpu and nef have been deleted to make lentiviral vectors safe as gene therapy vectors for human use. Lentiviral vectors provide several advantages for gene therapy. They integrate stably into chromosomes of target cells, which is required for long-term expression, and they do not transfer viral genes, therefore avoiding the problem of generating transduced cells that can be destroyed by cytotoxic T lymphocytes. In addition, lentiviral vectors have a relatively large cloning capacity, sufficient for most envisioned clinical applications. Furthermore, lentiviruses (in contrast to other retroviruses) are capable of transducing non-dividing cells. This is very important in the context of gene therapy for some tissue types, particularly hematopoietic cells, brain, liver, lungs and muscle. For example, vectors derived from HIV-1 allow efficient in vivo and ex vivo delivery, integration and stable expression of transgenes into cells such a neurons, hepatocytes, and myocytes.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′LTR's serve to promote transcription and polyadenylation of the virion RNA's. The LTR contains all other cis-acting sequences necessary for viral replication. Lentiviruses also have additional genes, including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

A number of different lentiviral vectors, packaging cell lines and methods of generating lentiviral gene therapy vectors are known in the art.

Also provided herein are isolated cells comprising the nucleic acid molecules or vectors disclosed herein. For example, the isolated cell can be a cell (or cell line) appropriate for production of lentiviral gene therapy vectors, such as a packaging cell line. Exemplary cell lines include HeLa cells, 293 cells and PERC.6 cells.

In some embodiments, the recombinant lentivirus compositions contain a pharmaceutically acceptable excipient as described above.

Also provided herein is a method of treating immunological abnormalities, such as myeloid dysfunction, in a subject diagnosed with GSD-Ib. In some embodiments, the method includes obtaining bone marrow cells from the subject, transducing the bone marrow cells ex vivo with a recombinant virus disclosed herein, and infusing the transduced bone marrow cells into the subject. In some examples, the recombinant virus is a rAAV or recombinant lentivirus.

In one aspect, the subject is a subject in need of treatment or prevention of glucose storage disease. In an aspect, the subject has an abnormal metabolic phenotype or profile. In other embodiments, the subject has neutropenia and/or myeloid dysfunction. In an embodiment, the subject is a newborn or neonate.

The therapeutic composition can be administered as a component of a pharmaceutical formulation. The pharmaceutical formulation can further include a pharmaceutically acceptable carrier. In an embodiment, the method of preventing or treating glycogen storage disease caused by a deficiency in either G6PT or G6Pase α, or G6Pase β comprises administering an effective amount of a pharmaceutical formulation comprising the therapeutic composition to a subject.

The pharmaceutical formulations disclosed herein can include a pharmaceutically acceptable carrier. As used herein, the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals. The term “pharmaceutically acceptable carrier” refers to an excipient, diluent, preservative, solubilizer, emulsifier, adjuvant (also referred to as immunological adjuvant), and/or vehicle with which the present antibody or fragment is administered. Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy)ethoxy]ethyl (E)-octadec-9-enoate), disodium edetate dehydrate, sucrose, monobasic sodium phosphate monohydrate, and dibasic sodium phosphate dihydrate. Except insofar as any conventional media or agent is incompatible with the gene therapy, such use in the pharmaceutical formulation is contemplated.

The pharmaceutical formulations are formulated to be suitable for the intended route of administration to a subject. For example, the pharmaceutical formulation may be formulated to be suitable for intravenous, oral, intraperitoneal, intranasal, intratracheal, subcutaneous, intramuscular, topical, intradermal, transdermal or pulmonary administration. In an embodiment, the gene therapy or pharmaceutical formulation comprising the gene therapy can be formulated so that it is suitable for administration to a human subject. In an embodiment, the pharmaceutical formulation comprising gene therapy is formulated so that it is suitable for subcutaneous, oral, intra-muscular, intra-nasal, intravaginal, or mucosal administration to a human subject.

Pharmaceutical formulations disclosed herein can comprise a stabilizer to prevent loss of activity or structural integrity of the gene therapy, oxidation or aggregation over a period of time during storage and transportation prior to use. The pharmaceutical formulation can comprise one or more of any combination of salts, surfactants, pH and tonicity agents such as sugars can contribute to overcoming aggregation problems. Where a pharmaceutical formulation of the present invention is formulated for injection, it is desirable to have a pH value in an approximately neutral pH range, and it is also advantageous to minimize surfactant levels to avoid bubbles in the formulation which are detrimental for injection into a subject. The pharmaceutical formulation can be in liquid form and can stably support high concentrations of bioactive antibody in solution. In an embodiment, the pharmaceutical formulation is suitable for intravenous, oral, intramuscular, intraperitoneal, intradermal, and/or subcutaneous injection. In an embodiment, the pharmaceutical formulation is in liquid form and has a minimized risk of bubble formation and anaphylactoid side effects. The pharmaceutical formulation can have a pH of 6.8 to 7.4. The pharmaceutical formulation can be isotonic.

In an embodiment the pharmaceutical formulation comprising the gene therapy described herein is substantially pure with regard to the gene therapy. A composition or pharmaceutical composition comprising the gene therapy, described herein is “substantially pure” with regard to the gene therapy when at least 60% to 75% of a sample of the composition or pharmaceutical composition exhibits a single species of the gene therapy. A substantially pure composition or pharmaceutical composition comprising the gene therapy, described herein can comprise, in the portion thereof which is the gene therapy, 60%, 70%, 80% or 90% of the gene therapy, more usually about 95%, and preferably over 99%. Purity or homogeneity may be tested by a number of means well known in the art, such as polyacrylamide gel electrophoresis or HPLC.

The gene therapy product, and/or pharmaceutical formulation described herein can also be lyophilized and/or freeze dried and subsequently reconstituted for use, or provided in any suitable form including, but not limited to, ingestable product or food, injectable solutions or inhalable solutions, gel forms and tablet forms.

The gene therapy can be administered as a single dose, or as a plurality of doses separated by a defined period of time. For example, a first dose of gene therapy can be at a neonatal stage, followed by a subsequent second dose a period to 2 to 6 weeks, or 2 to 4 weeks after the first dose, or later.

It is understood that aspects and embodiments of the invention described herein include “consisting” and/or “consisting essentially of” aspects and embodiments.

The terms “a” and “an” do not denote a limitation of quantity, but rather denote the presence of at least one of the referenced item. The term “or” means “and/or”. As used herein, the term “and/or” includes any and all combinations of one or more of the associated listed items. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

This disclosure is further illustrated by the following the Experimental Details, which are non-limiting.

### Experimental Details

### Materials and Methods Part 1. In Vitro Development

Cell line and culture conditions: Human hepatoma G2 (HepG2), Human embryonic kidney 293T (HEK 293) cells, and Human leukemia cells (HL-60) were purchased from ATCC and maintained in Dulbecco's modified Eagle medium (DMEM/High-Glucose) supplemented with 10% fetal bovine serum and 1% Penicillin-Streptomycin. All cells were incubated at 37° C., 5% CO2 incubator.

Transfection conditions: For promoter evaluation, we constructed TurboGFP expressing plasmids each with a different human G6PT, 1.8 kb, 2.3 kb, 1 kb-A, and 1 kb-B promoter sequence (FIG. 1A). For transfection, 5×10⁵ of HepG2 or HEK293 cells were transfected by Lipofectamine LTX™ with Plus Reagent (ThermoFisher Scientific™) using 2.5 ug of plasmid DNA according to the manufacturer's protocol. For HL-60 cells, 0.8-1.5×10⁵ cells and 1 μg of plasmid DNA were used for transfection by Neon Electroporation system (ThermoFisher Scientific™) according to the manufacturer's protocol. 36-48 hours after the transfection, the mean fluorescence of GFP signal was measured from the transfected cells by Attune NXT™ Flow Cytometer (ThermoFisher Scientific™).

B. Evaluation of Codon Optimized hG6PT

Cell line use and transfection: To evaluate codon optimized human G6PT, we constructed an AAV vector expressing wild type human G6PT (hSLC37A4) gene and codon-optimized human G6PT gene driven by 1 kb-A promoter from the promoter comparison study. We have named the 1 kb-A promoter sequence as glucose-6-phosphatase transporter promoter (GTP). Therefore, the names of these plasmid vectors are pAAV-GTP-hG6PT and pAAV-GTP-co-opt-hG6PT. For transfection, 1.5×106 of HepG2 or HEK293 cells were transfected by Lipofectamine™ LTX with Plus Reagent (ThermoFisher Scientific™) using 7.5 ug of plasmid DNA according to the manufacturer's protocol.

Western blot: Western-blots were analyzed using the ChemiDoc (Bio-Rad Inc™). The antibodies used were β-actin (sc-47778) from Santa Cruz Biotechnology™; Anti-SLC37A4 (ab80463) from Abcam®.

### Materials and Methods Part 2. In Vivo Evaluation

AAV production: The vector, pAAV-GTP-co-opt-hG6PT was packaged in AAV8, AAV9 or AAV2-quadYF serotypes and ultra-purified using Vector Builder service (Vector builder Inc.)

General mice maintenance and identification of GSD-Ib mice (G6pt^{-/-} mice): G6pt−/− mice were obtained from NICHD/NIH. All animal studies were performed in accordance with the guidelines of the Institutional Animal Care and Use Committee of The University of Connecticut Health Center. All mice were maintained in a pathogen-free animal facility at 22-24° C. under the 12:12 light: dark cycle. Standard rodent chow (Envigo™, USA) and water were provided ad libitum. Newborn GSD-Ib mice can be identified easily within 3 days after birth by appearance including their smaller body size and hepatomegaly.

AAV administration: AAV vectors were administered to the G6pt−/− mice in single dose or multidose. For single-dose and the first dose of two-dose group, 2-3×10¹³ vg/kg dose of AAV8 serotype GTP-co-opt-hG6PT was injected neonatally (withing 3 days after birth) via the temporal vein. For the age-12 weeks of multidose group, 1 or 3×10¹³ vg/kg dose of AAV8, AAV9, or AAV2-quadYF serotype GTP-co-opt-hG6PT were injected via the retro-orbital sinus. Age-matched littermate G6pt^{+/+} or G6pt^{+/-} mice were used as controls.

Single-dose survival test: To rescue the extremely low survival rate of G6pt^{-/-} mice and to determine initial in vivo efficacy of the vector, we measured survival rate for the group receiving single-dose at newborn (NB) with AAV8-GTP-co-opt-hG6PT (2-3×10¹³ vg/kg). until 40 weeks.

Two-dose survival test (up to 24 weeks): Based on the result of the single dose survival test, the two-dose group received a 2nd dose at 12 weeks. At 24 weeks (12 weeks after 2nd dose), we measured survival rates.

Fasting glucose test: To evaluate the efficacy of the vector in rescuing hypoglycemia during fasting, we performed fasting glucose tests for 24 hours. During the fasting, we measured blood glucose at 0, 2, 4, 6, 8, and 24 hours. Blood glucose levels were measured using a blood glucose meter and glucose cuvettes (HemoCue® Glucose 201 System; HemoCue™, Brea, CA, USA)

Serum collection and analysis (4 w, 12 w, 18 w, and 24 w or older): G6pt^{-/-} mice showed disturbed blood metabolic abnormalities including triglycerides, cholesterol, lactic acid, and uric acid (L. Y. Chen et al., Impaired glucose homeostasis, neutrophil trafficking and function in mice lacking the glucose-6-phosphate transporter. Hum Mol Genet 12, 2547-2558 (2003)), we measured these metabolites from the serum of vector administrated G6pt−/− mice and them compared age-matched control group. To determine triglyceride, cholesterol, lactic acid and uric acid levels in blood, serum was collected from each mouse group at age 4, 12, 18, and 24<weeks. Total cholesterol and uric acid were analyzed using kits obtained from ThermoFisher Scientific™. Triglycerides were measured with a Serum Triglyceride Determination Kit (Sigma-Aldrich™, USA) and lactate measured with a colorimetric/fluorometric kit (Biovision, USA). For each respective kit, samples were prepared and analyzed according to manufacturer's instructions using the SpectraMax® i3x (Molecular Devices, USA).

Blood absolute neutrophil counts: To measure absolute neutrophil counts, mouse peripheral blood samples were collected at age 4, 12, 18, and 24<weeks. The collected blood samples were treated with ammonium-chloride-potassium (ACK) lysis buffer (ThermoFisher Scientific™) to remove red blood cells. The resulting leukocytes were counted and stained with mouse Fluorescein isothiocyanate (FITC)-conjugated Lymphocyte antigen 6 complex locus G6D (Ly6G) antibody and mouse phycoerythrin-cyanine5.5 (PE-Cy5.5)-conjugated integrinalpha M (CD11b) antibody (eBioscience®) for 20 min at 4° C. in the dark. Cells were analyzed with an Attune™ NxT Flow Cytometer (ThermoFisher Scientific™).

Terminal analysis (fasted 6 hours, at >24 weeks): Once the mice were older than 24 weeks, the AAV vector administered mice and control mice were fasted 6 hours and sacrificed to collect tissue samples including, liver, kidney, intestine, muscle, heart, lung, spleen, bone marrow, and testis/ovary. To evaluate the status of hepatomegaly in the AAV vector administrated group, liver/kidney weight and body weight were measured to compare the percentage of the organ weight to body weight.

Vector Copy number analysis: To evaluate vector distribution and estimate efficacy of the vector, we measured vector copy numbers from the collected tissue samples. Genomic DNA was isolated and real-time quantitative PCR (RT-qPCR) was performed to measure Cq value with a primer pair, forward: GTGATCTTCAGCGCCATGTT (SEQ ID NO: 7) and reverse: GAACTTGCTGATGGCGTAGG (SEQ ID NO:8), that detects codon optimized human G6PT from the genomic DNA. And then the copy number was calculated by standard curve and equation generated by serial dilution of plasmid vector of pAAV-GTP-co-opt-hG6PT.

mRNA expression confirmation: To confirm in vivo gene expression, total RNA was isolated from the livers with TRIzol™ Reagent (Invitrogen™, USA) and RNeasy® Mini Kit (QIAGEN, USA) according to the manufacturer's instructions. For cDNA synthesis, iScript™ gDNA Clear cDNA Synthesis Kit (Bio-Rad Laboratories, USA) was used according to the manufacturer's instructions. The mRNA expression was quantified by CFX96™ real-time PCR detection system (Bio-Rad Laboratories, USA). Data were analyzed using the CFX Maestro™ software (Bio-Rad Laboratories) and normalized to the mouse ribosomal protein L19 (Rp119) mRNA expression.

Protein expression confirmation: To confirm in vivo protein expression, we used western-blots analysis using the ChemiDoc™ (Bio-Rad Inc) with homogenized liver tissue samples from the AAV vector-administrated group. The antibodies used were β-actin (sc-47778) from Santa Cruz® Biotechnology; Anti-SLC37A4 (ab80463) from Abcam®.

### Example 1: Construction of pAAV-GTP-Co-Opt-hG6PT Gene Therapy Vector

The vector components are shown in Table 1 below. The vector contains codon optimized human SLC37A4 sequence driven by 1 kb upstream sequence (−1 to −1000 bp) of human SLC37A4 gene.

TABLE 1
VECTOR COMPONENTS OF PAAV-GTP-CO-OPT-HG6PT

| Name | Position | Size (bp) | Type | Description | Application notes |
|---|---|---|---|---|---|
| 5′ ITR | 1-141 | 141 | ITR | AAV 5′ inverted terminal | Allows rescue of |
| | | | | repeat (functional | virus from |
| | | | | equivalent of wild-type 5′ | recombinant |
| | | | | ITR) | plasmid and |
| | | | | | replication of the |
| | | | | | viral genome; this |
| | | | | | ITR is identical to |
| | | | | | that of the wild- |
| | | | | | type AAV2 |
| | | | | | genome. |
| GTP | 169-1168 | 1000 | Promoter | Human G6PT 5′ Flanking | |
| 1 kb-A | | | | Sequence −1000 to −1 | |
| human | | | | | |
| G6PT | | | | | |
| promoter | | | | | |
| Kozak | 1193-1198 | 6 | Miscellaneous | Kozak translation | Facilitates |
| | | | | initiation sequence | translation |
| | | | | | initiation of ATG |
| | | | | | start codon |
| | | | | | downstream of the |
| | | | | | Kozak sequence. |
| Co-opt | 1199-2488 | 1290 | ORF | Codon optimized hG6PT | Ref sequence |
| hG6PT | | | | variant 1 | Transcript ID: |
| | | | | | NM_001164277.2, |
| | | | | | Ref sequence |
| | | | | | Protein ID: |
| | | | | | NP_001157749.1 |
| WPRE | 2519-3116 | 598 | Miscellaneous | Woodchuck hepatitis | Enhances virus |
| | | | | virus posttranscriptional | stability in |
| | | | | regulatory element | packaging cells, |
| | | | | | leading to higher |
| | | | | | titer of packaged |
| | | | | | virus; enhances |
| | | | | | higher expression |
| | | | | | of transgenes. |
| BGH pA | 3147-3354 | 208 | PolyA_signal | Bovine growth hormone | Allows |
| | | | | polyadenylation signal | transcription |
| | | | | | termination and |
| | | | | | polyadenylation of |
| | | | | | mRNA transcribed |
| | | | | | by Pol II RNA |
| | | | | | polymerase. |
| 3′ ITR | complement | 141 | ITR | AAV 3′ inverted terminal | Allows rescue of |
| | (3362-3502) | | | repeat | virus from |
| | | | | | recombinant |
| | | | | | plasmid and |
| | | | | | replication of the |
| | | | | | viral genome; this |
| | | | | | ITR is identical to |
| | | | | | that of the wild- |
| | | | | | type AAV2 |
| | | | | | genome. |
| Ampicillin | 4419-5279 | 861 | ORF | Ampicillin resistance gene | Allows E. coli to |
| | | | | | be resistant to |
| | | | | | ampicillin. |
| pUC ori | 5450-6038 | 589 | Rep_origin | pUC origin of replication | Facilitates plasmid |
| | | | | | replication in E. |
| | | | | | coli; regulates |
| | | | | | high-copy plasmid |
| | | | | | number (500-700). |

### Example 2: 5′-Flanking Region of the Human SLC37A4 (G6PT) Gene, from −1 to −1000 Induced the Highest GFP Expression in Cell Line Tests

For promoters, we compared 4 different upstream sequences to express GFP signals in the different cell lines.

Based on our investigation of the 5′ upstream sequence of the transcription starting site of human G6PT gene, we found:
TATA-box [p-value=0.01]: at −520, −1191, −1801, −1875
Initiator [p-value=0.01]: at −26, −180, −272, −812, −888, −904, −923, −1211, −1237, −1294, −1309, −1416, −1496, −1620, −1632, −1841, −1945
GC-box [p-value=0.001]: at 32, −427, −963, −1425, −1904
CCAAT-box [p-value=0.01]: at 88, −133, −149, −176, −206, −282, −378, −469, −662, −748, −869, −1149, −1207, −1313, −1487, −1514

Analysis of ChIP-Seq data showed that both H3K4mel (which is known to be associated with enhancer element) and RNA polymerase II recruiting sites are located within 1 kb upstream of the transcription starting site of hG6PT.

Collectively, and considering packaging size of AAV vector, we chosen 1 kb-A, −1000 to −1 of 5′ flanking sequence of hG6PT.

In addition, we choose to compare other potential promoter sequences in fragments 1.8 kb, 2.3 kb, and 1 kb-B promoter to 1 kb-A promoter.

Although we had chosen 1 kb-A sequence after analyzing promoter motifs, as mentioned above, ChIP-seq data, the ChIP-seq data still showed additional H3K4mel, RNA polymerase II recruiting sites, initiator, GC-box, and CCAAT-box located downstream of transcription starting site of hG6PT. In addition, as exon 1, exon 2 and half of exon3 are in the 5′untranlated region (UTR), we also needed to compare these sequences as potential promoters, so we chose 1.8 kb, 2.3 kb upstream of the translation start site as comparison sequences. As we found CpG island further upstream, we also included 1 kb-B (−3700 to −2700) as another potential promoter sequence for comparison.

To construct GFP expressing-promoter testing plasmids, we used Vector Builder Inc for plasmid construction. Briefly, the 1.8 kb, 2.3 kb, 1 kb-A, and 1 kb-B sequences were each cloned and inserted into a promoter testing plasmid (vector builder Inc) containing TurboGFP as a reporter gene.

The nucleotides −1000 to −1 region of the human SLC37A4 5′-flanking region presumably includes, distal enhancers, RNA polymerase II binding sites and multiple transcriptional motifs which could control the gene expression of human SLC37A4 gene. Human SLC37A4 is known to have a ubiquitous gene expression pattern. To investigate the gene expression pattern and compare the strength of the promoter sequences, we constructed a plasmid vector that expresses GFP under control of a the human SLC37A4 promoter at nucleotides −1000 to −1 and compared it with other candidate sequences from the human SLC37A4 5′-flanking region. As a result, nucleotides −1000 to −1 (1 kb A promoter) showed the highest gene expression.

### Example 3: Codon Optimization of Human SLC37A4 Achieved Significantly Increased Protein Production

In order to develop robust expression, we evaluated whether codon optimization could increase human SLC37A4 protein production. We constructed two AAV vectors, one with wild type human SLC37A4 and another with codon optimized human SLC37A4, both under control of nucleotides −1000 to −1 from 5′-flanking region of human SLC37A4 gene (hereafter referred to as “GPT promoter”). Using the imageJ program to quantify the western blots of FIG. 2A-B, Set 1-3, we compared the expression of these two AAV vectors after transfection in HepG2 and HEK293 cell lines, and found that the vector construct that contains codon optimized human SLC37A4 gene showed significantly higher protein expression. The codon-optimized human SLC37A4 gene vector showed 3.3-fold higher protein production and 9.5-fold higher protein production in the HepG2 and HEK293 cell line, respectively (FIGS. 2A and 2B).

### Example 4: pAAV-GPT-Co-Opt-hG6PT Treated G6PT−/− Mice Showed Improved Survival Rates in In Vivo Assays

To evaluate the gene therapy vector construct, pAAV-GPT-co-opt-hG6PT in vivo, we packaged the vector into three AAV serotypes, AAV8 or AAV9, and AAV2-quadYF. We chose these three vectors in order to avoid potential neutralizing antibody against the AAV serotype used in the first administration (newborn administration): AAV8 was used for NB injection, and AAV9 and AAV2-quadYF were used in the later-administered dose thereby avoiding potential neutralizing antibody against AAV8. For the single dose survival test, we injected AAV8-GPT-co-opt-hG6PT at age day 2-5 with 1×10\hat{ }13 vg/kg (vector genome per kilogram) of body weight. For the double dose treatment group, we injected AAV8-GPT-co-opt-hG6PT at age day 2-5 with 1×10¹³ vg/kg of body weight, and then we administered a 2nd dose with 1×10¹³ vg/kg or 3×10¹³ vg/kg at 12 weeks of age.

To evaluate the effectiveness of the vector therapy in improving fasting hypoglycemia, a fasting blood glucose test was performed at age 4 weeks, 12 weeks, 18 weeks (6 weeks after 2nd dose), and 24 weeks (12 weeks after 2nd dose). Blood metabolites including non-fasted blood glucose, triglycerides, cholesterol, lactic acid, and uric acid were measured at age 4 weeks, 12 weeks, 18 weeks (6 weeks after 2nd dose), and 24 weeks (12 weeks after 2nd dose). Body weight growth curves were measured.

For terminal analysis, liver weight, vector copy number, gene expression, and human SLC37A4 protein expression were measured.

To evaluate the effects on neutropenia, absolute neutrophils were counted along with fasting blood glucose tests at age 4 weeks, 12 weeks, 18 weeks (6 weeks after 2nd dose), and 24 weeks (12 weeks after 2nd dose).

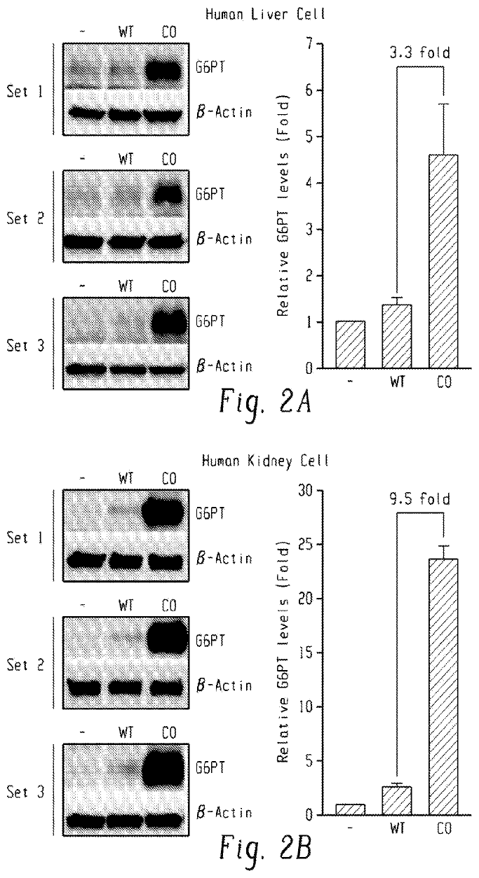

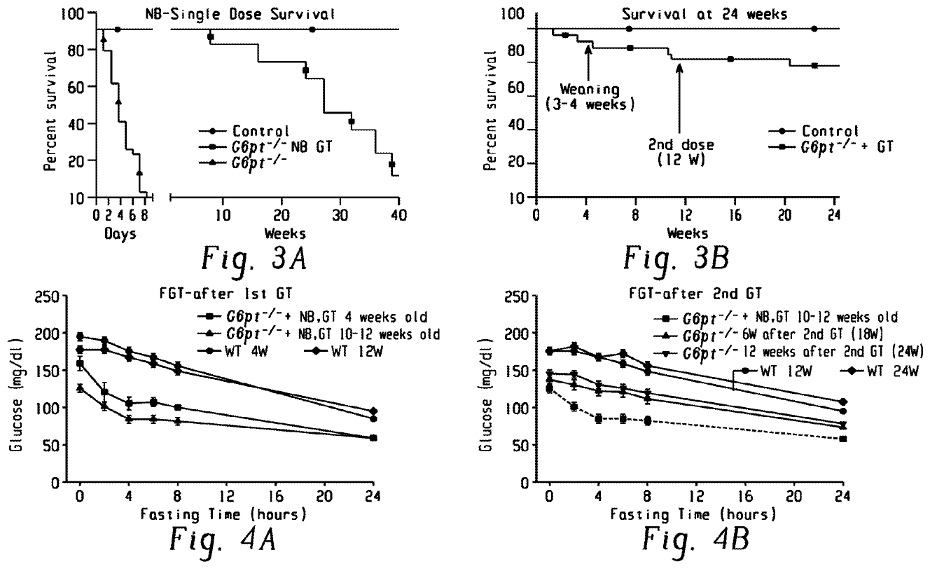

The survival rate of the single dose and double dose groups were measured. Without glucose supplement therapy the survival rate of G6pt−/− mice is extremely low and more than 50% died within 5 days after birth (FIG. 3A). Therefore, gene therapy targeting newborn (NB) G6pt−/− mice is essential. In the NB-Single dose treatment group with AAV8-GPT-co-opt-hG6PT vector administration, 100% of the treated G6pt−/− mice survived until 8 weeks and more than 80% of treated G6pt−/− mice survived until 24 weeks (FIG. 3A). The survival rate started to decrease after 24 weeks post vector infusion. It has previously been shown in a GSD-Ia gene therapy study (Yiu et al. 2009) that the activity from gene therapy decreased drastically during growth, especially in the liver. However, surprisingly, the therapy vector AAV GPT-co-opt-hG6PT showed a remarkable improvement and enabled survival of G6pt−/− mice to adult age (12 weeks), thereby overcoming the vector dilution effect seen by other investigators.

For the double dose treatment group, we administrated the vector at 12 weeks of age based on single dose study result. After the second dose, the survival rate of treated G6pt−/− mice showed minimal changes from 80% through 24 weeks whereas the single dose survival at 24 weeks was close to 50% (FIG. 3B). Therefore, the therapy vector treatment enabled adult-age infusion that is long-lasting without vector dilution due to liver growth.

### Example 5: pAAV-GPT-Co-Opt-hG6PT Treated G6PT−/− Mice Maintained Normal Blood Glucose Level During 24 Hrs of Fasting Test

To evaluate the efficacy of the vector therapy to improve fasting hypoglycemia, fasting blood glucose tests were performed at age 4 weeks and 12 weeks after 1st dose of gene therapy at NB (FIG. 4A). The fasting blood glucose tests were also performed at age 18 weeks (6 weeks after 2nd dose) and 24 weeks (12 weeks after 2nd dose). At 4 weeks after the 1st dose, the treated G6pt−/− mice were able to fast 24 hours without hypoglycemia. The average blood glucose level at 6-8 hours of fasting was over 100 mg/dl (within normal range of blood glucose level). There was decrease in fasting blood glucose levels from 4 to 12 weeks of age along with body weight increase, however, the treated G6pt−/− mice were able to fast 24 hours without hypoglycemia and the average 6-8 hours of fasting blood glucose levels was over 80 mg/dl (within normal range of blood glucose level). Six weeks after 2nd dose, the treated G6pt−/− mice showed improved fasting glucose levels from 12 weeks and then, the levels of glucose increased and were maintained above 120 mg/dl at 6-8 hours of fasting until 24 weeks old (12 weeks after 2nd dose) (FIG. 4B).

### Example 6: Growth Profile of pAAV-GPT-Co-Opt-hG6PT Treated G6PT−/− Mice

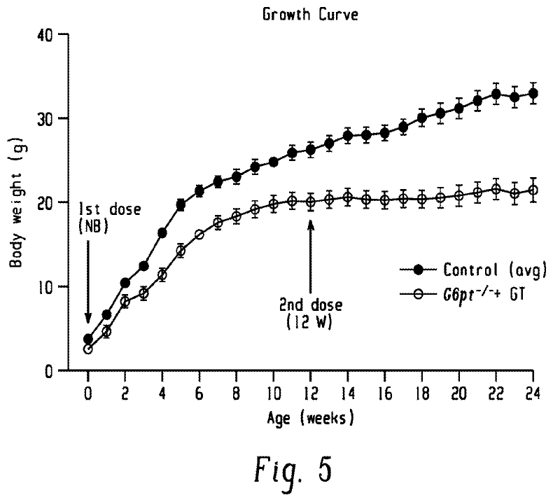

Body weight was recorded weekly to track the growth rate of gene therapy-treated G6pt−/− mice. At 2-3 days after birth, G6pt−/− mice can be distinguished by their appearance such as smaller size and hepatomegaly. While G6pt−/− mice failed to survive beyond a week without intervention such as glucose supplement, the gene therapy vector-treated G6pt−/− mice survived and grew until 10 weeks, with the growth rate plateauing after the second dose (FIG. 5).

Results from a previous gene therapy study using a viral promoter showed increased body weight in gene therapy-treated G6pt−/− mice compared to WT control mice, and the mice developed severe hepatomegaly and HCA/HCC (Yiu et al., 2010, Mol. Ther. 18:1076-1084). These results proved that increased body weight over a certain level for G6pt−/− mice was not a good sign for metabolic control of G6pt−/− mice. Another GSD-Ib gene therapy approach showed that lower body weight was associated with higher hepatic G6PT activity in G6pt−/− mice, with lower liver weight with less fat accumulation (Kwon et al., 2017 Hum Mol Genet 26:4395-4405).

### Example 7: Serum Metabolite Profile of pAAV-GPT-Co-Opt-HG6PT Treated G6PT−/− Mice

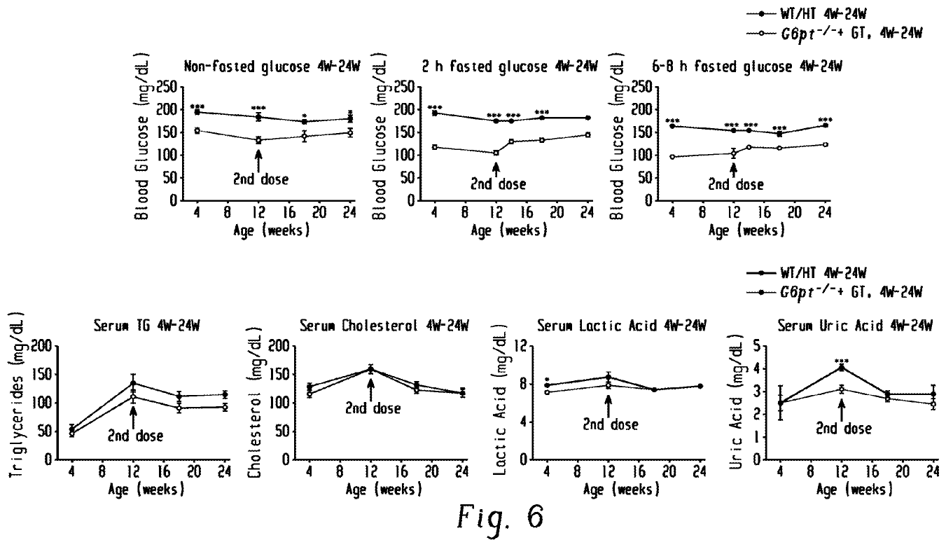

Blood glucose and key metabolites were monitored at age 4 weeks, 12 weeks, 18 weeks (6 weeks after 2nd dose), and 24 weeks (12 weeks after 2nd dose). According to the fasting glucose test, this gene therapy vector is capable of restoring the ability of treated G6pt−/− mice to maintain normal blood glucose level for up to 24 hours. However, normally, 24 hours of fasting is not relevant to normal life. Therefore, in this figure, we aligned data from the non-fasted, 2 hours fasted, and 6-8 hours of fasting groups. In these plots, we found that the blood glucose levels of non-fasted, 2 hours fasted, and 6-8 hours of fasting were increased after 2nd dose and maintained until 24 weeks (FIG. 6).

The blood metabolites remained in normal ranges until 24 weeks with minimal difference from WT control.

### Example 8: Body Weight (BW), Liver Weight/Body Weight (LW/BW), and Kidney Weight/Body Weight (KW/BW)

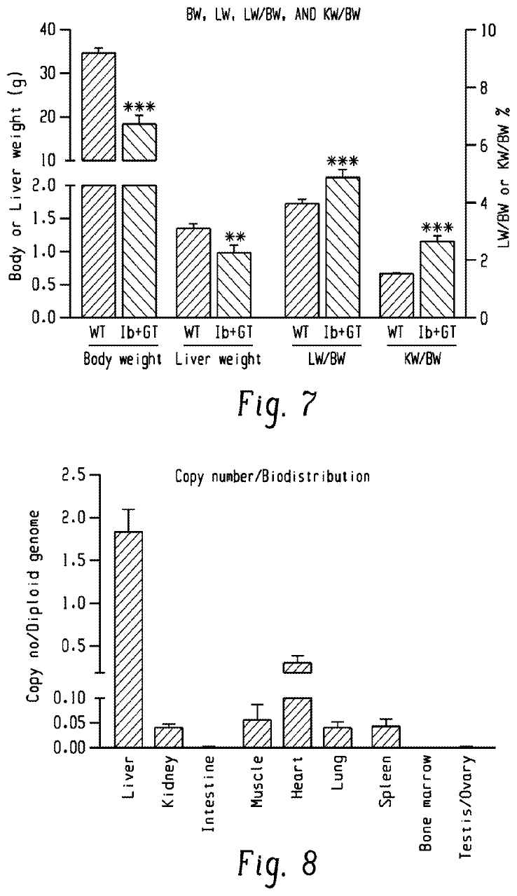

As described previously, body weight of gene therapy treated G6pt−/− mice remained lower than WT. Although gene therapy-treated G6pt−/− mice (1st or 2nd doses) showed elevated LW/BW than WT, the average percentage of LW/BW in this study was improved from previous publication (Kwon et al. 2017, Hum. Mol. Genet. 26:4395-4405). In addition, average liver weight of the gene therapy-treated G6pt−/− mice was even lower than WT (FIG. 7). Kidney weight remained significantly elevated in the gene therapy-treated G6pt−/− mice (FIG. 7).

### Example 9: Gene Copy Number Distribution

Copy number analysis revealed that the highest vector copy number existed in liver (FIG. 8). Average copy number in a diploid genome of a liver cell was 1.82 copies per genome. We also found a significant copy number in the heart (0.31 copies per a genome). From 0.03 to 0.05 copies were found in other tissues including kidney, muscle, lung, and spleen. Vector copy number from intestine, bone marrow, and reproductive organs were not significant.

### Example 10: In Vivo Gene Expression (mRNA and Protein) Confirmation

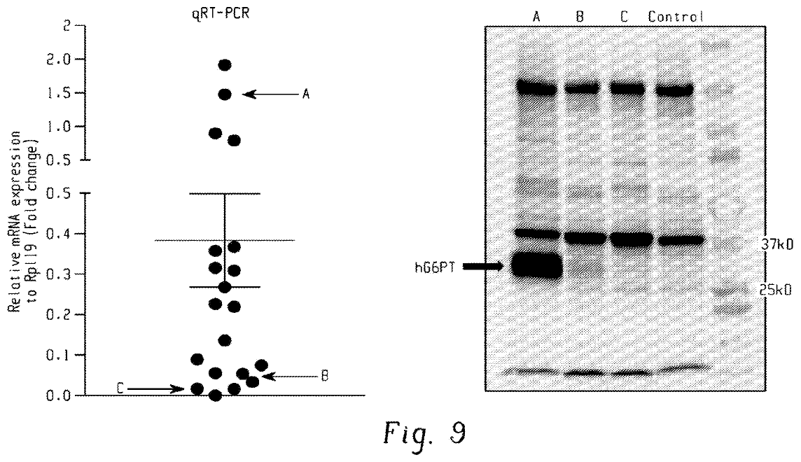

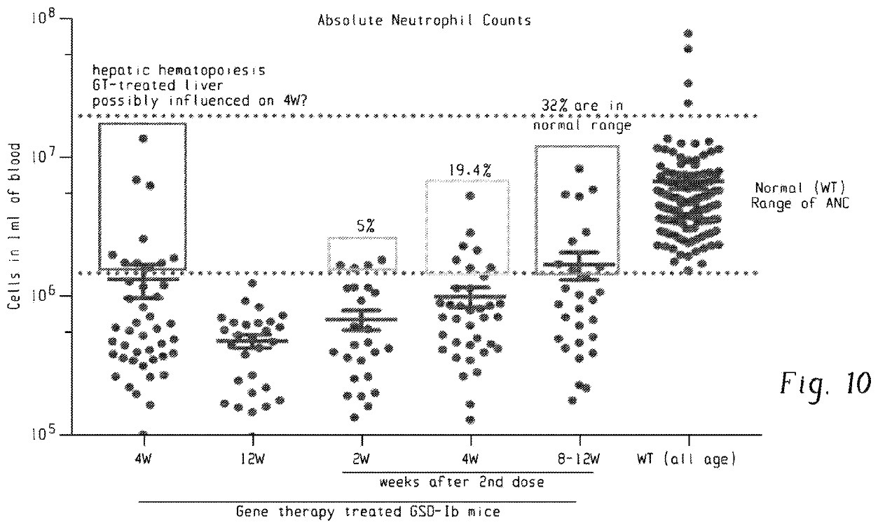

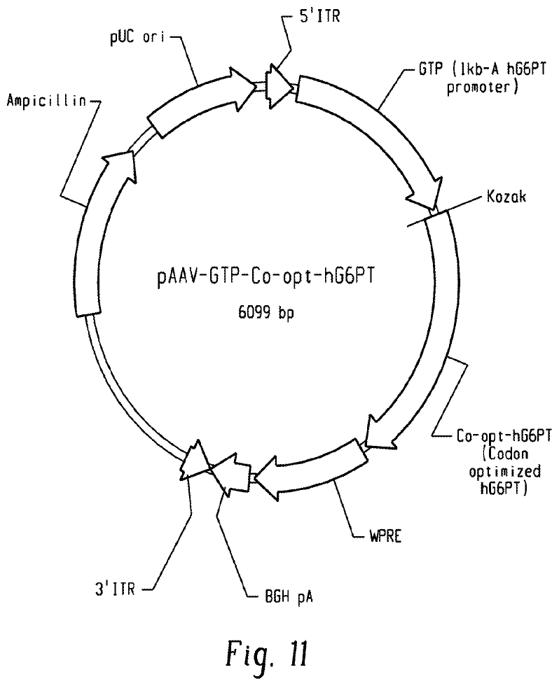

We confirmed that this vector is capable of expressing co-opt-hG6pt in various cell lines from different origins. We used relative mRNA levels measured by qRT-PCR (FIG. 9, high level indicated at A, and low level indicated at B and C), and protein level in a western blot (FIG. 9, high level indicated in lane A, and low level indicated in lanes B and C). qRT-PCR and western blot analysis showed that this vector is successfully expressed in liver (FIG. 9). Data shows that the protein expression amount and mRNA expression pattern is similar. These results confirm that the vector can be expressed in vivo both as mRNA and as protein, and has in vivo efficacy, effectively rescuing the subject from hypoglycemia and normalizing metabolites, improving survival rate and reducing or eliminating disease other phenotypes such as hepatomegaly.

### Example 11: pAAV-GPT-Co-Opt-hG6pt Treated G6PT−/− Mice Showed Improvement in Neutropenia

The pAAV-GPT-co-opt-hG6pt gene therapy vector has a positive effect on absolute neutrophil counts (ANC) in gene therapy-treated G6pt−/− mice. From white blood cell (WBC) analysis, we found that 24% of 4-week old G6pt−/− mice receiving gene therapy (G6pt−/−+GT) have ANC at a normal range. However, this effect disappeared when the G6pt−/−+GT mice reached 12 weeks of age. However, after a 2nd dose of gene therapy, a few of the G6pt−/−+GT mice showed a normal range ANC. The percentage of mice with a normal range increased at 2 weeks (5%), 4 weeks (19.4%) and 8-12 weeks (32%) after 2nd dose of gene therapy.

### DISCUSSION

The new gene therapy vector, pAAV-GTP-co-opt-hG6pt showed strong gene expression, and achieved several fold higher gene expression in human liver and kidney origin cell lines.

Neonatal infusion of AAV8-GTP-Co-opt-hG6pt showed a dramatic increase in survival rates and corrected metabolic abnormalities until 24 weeks with sustained fasting glucose levels.

The 2nd dose improved multiple factors associated with metabolic abnormalities and showed that it has potential to correct neutropenia in GSD-Ib. pAAV-GTP-co-opt-hG6pt is a promising gene therapy vector for GSD-Ib.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A recombinant nucleic acid molecule comprising SEQ ID NO:1.

2. The recombinant nucleic acid molecule of claim 1, wherein said molecule comprises nucleotides 1-3502 of SEQ ID NO:1.

3. The recombinant nucleic acid molecule of claim 1, wherein said molecule comprises nucleotides 169 to 3354 of SEQ ID NO:1.

4. The recombinant nucleic acid molecule of claim 1, wherein said molecule comprises nucleotides 169 to 1290 of SEQ ID NO: 1.

5. A recombinant nucleic acid molecule of claim 1, wherein said molecule comprises nucleotides 169-1168 of SEQ ID NO:1.

6. A vector comprising the recombinant nucleic acid of claim 1.

7. The vector of claim 6, wherein the vector is an adeno-associated virus (AAV) vector.

8. The vector of claim 7, wherein the AAV vector is an AAV serotype 8 (AAV8) vector, serotype 9 (AAV9), or serotype QuadFY (rAAV-quadFY).

9. A recombinant AAV (rAAV) comprising the recombinant nucleic acid molecule of claim 1.

10. The rAAV vector of claim 9, wherein the vector is rAAV8, rAAV9, or rAAV-quadFY.

11. The vector of claim 6, which is a lentivirus vector.

12. A composition comprising the rAAV of claim 9 in a pharmaceutically acceptable carrier.

13. The composition of claim 12 formulated for intravenous administration.

14. A method for treating a subject diagnosed with a glycogen storage disease, comprising selecting a subject with glycogen storage disease type Ib (GSD-Ib) and administering to the subject a therapeutically effective amount of the rAAV of claim 9.

15. The method of claim 14, wherein the rAAV is administered intravenously.

16. The method of claim 14, comprising administering about 1×10¹¹ to about 1×10¹⁴ vector genes (vg)/kg of the rAAV per dose, about 1×10¹² to about 1×10¹⁴ vg/kg of the rAAV per dose, or about 5×10¹² to about 5×10¹³ vg/kg of the rAAV per dose.

17. The method of claim 14, wherein administering the rAAV comprises administration of a single dose of the rAAV or of multiple doses of the rAAV.

18. The method of claim 17, wherein a second dose is administered at a different time point from a first dose, determined by detecting in the subject a loss in a measure of metabolic abnormality correction chosen from the group consisting of fasting blood glucose, non-fasted blood glucose, triglycerides, cholesterol, lactic acid, and uric acid, liver weight, vector genome copy number, G6PT gene expression, and G6PT protein expression, and improved neutrophil number.

19. A method for reducing symptoms of a glycogen storage disease, comprising selecting a subject with glycogen storage disease type Ib (GSD-Ib) and administering to the subject a therapeutically effective amount of the rAAV of claim 9.

20. A method for improving a metabolic abnormality metabolic abnormality chosen from the group consisting of fasting blood glucose, non-fasted blood glucose, triglycerides, cholesterol, lactic acid, and uric acid, liver weight, vector genome copy number, G6PT gene expression, and G6PT protein expression, and improved neutrophil number, said method comprising selecting a subject with glycogen storage disease type Ib (GSD-Ib) and administering to the subject a therapeutically effective amount of the rAAV of claim 9.


SEQUENCE LISTING (8 sequences)

SEQ ID NO: 1 - DNA, 6099 bp, synthetic construct
  source 1..6099: mol_type = other DNA; note = pAAV-GTP-Co-opt-hG6PT vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgtttgttaggctatgttaccttctgttgaagctatgggcagggttaacatttgctctgtttctgagaacacagtcatcattgttgtaaaatagcatcactactgttactcctcacaactcttaactgggatcagtgaaaaccattgttgttgagaactcttatagaatttctgagctcttatctaaatcatctgttgagtcttcacaatgatctaccaggctggtaggaaacgggagctcatagagattaaccaaggtcatacagttagaatgctgtgggtctaggactagaatttgatatttactccaggccccgtctctgtccccaacaatctaaccacaagttaaaattccaaattgtgggctgggcgcggtggctcacgcctgtaatcccagcactttgggaggccgaggtgagcggatcacgaggtcaggagatcgagaccatcctggcaaacacggtgaaaccccgtctctactaaaaatacaaaaaattacccgggcgtggtggtgggcgcccgtagtcccagctactcgggaggctgaggcaggagaatggtgtgaacccgggaggaggagcttgcagtgagccgaaatcgcgccactgcactccagcctgggcgactgagcgagactgtttcaaaaaaaaaaaaaaagaaaaaaagaaaatactgtgtgttcctacaacacctttcccagactgtgagcaacttgggaacaatgattatcaccttagtgttcggtgtgttttgtagaggagggcactcactaaaaactggtttgttgaatgcaaccacttaagaattggacatgccgtgcccagcccctaaggggcgctatgctcgttcgtccaaggagcagcgtgaccagcgggagggagtaagtcgacgtccccggcatgtggggcggggaactccactggccggcaagcgaagcagcaggcgaagaccacgcccccgccggccgcgcttgcgcagccttcgctagccccgccccgtcctcaagtttgtacaaaaaagcaggctgccaccatggccgcccagggctacggctactacagaaccgtgatcttcagcgccatgttcggcggctacagcctgtactacttcaacagaaagaccttcagcttcgtgatgcccagcctggtggaggagatccccctggacaaggacgacctgggcttcatcaccagcagccagagcgccgcctacgccatcagcaagttcgtgagcggcgtgctgagcgaccagatgagcgccagatggctgttcagcagcggcctgctgctggtgggcctggtgaacatcttcttcgcctggagcagcaccgtgcccgtgttcgccgccctgtggttcctgaacggcctggcccagggcctgggctggcccccctgcggcaaggtgctgagaaagtggttcgagcccagccagttcggcacctggtgggccatcctgagcaccagcatgaacctggccggcggcctgggccccatcctggccaccatcctggcccagagctacagctggagaagcaccctggccctgagcggcgccctgtgcgtggtggtgagcttcctgtgcctgctgctgatccacaacgagcccgccgacgtgggcctgagaaacctggaccccatgcccagcgagggcaagaagggcagcctgaaggaggagagcaccctgcaggagctgctgctgagcccctacctgtgggtgctgagcaccggctacctggtggtgttcggcgtgaagacctgctgcaccgactggggccagttcttcctgatccaggagaagggccagagcgccctggtgggcagcagctacatgagcgccctggaggtgggcggcctggtgggcagcatcgccgccggctacctgagcgacagagccatggccaaggccggcctgagcaactacggcaaccccagacacggcctgctgctgttcatgatggccggcatgaccgtgagcatgtacctgttcagagtgaccgtgaccagcgacagccccaagctgtggatcctggtgctgggcgccgtgttcggcttcagcagctacggccccatcgccctgttcggcgtgatcgccaacgagagcgccccccccaacctgtgcggcaccagccacgccatcgtgggcctgatggccaacgtgggcggcttcctggccggcctgcccttcagcaccatcgccaagcactacagctggagcaccgccttctgggtggccgaggtgatctgcgccgccagcaccgccgccttcttcctgctgagaaacatcagaaccaagatgggcagagtgagcaagaaggccgagtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggggtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 2 - DNA, 1001 bp, Homo sapiens
  source 1..1001: mol_type = genomic DNA; note = gene glucose-6-phosphate transporter sequence from -3700 to -2700
tggcgccatctcagctcactgcaacttctgcctcccaggttcaagtgattctcctgcctcagcctcctgattagctgggattacaggtgtacaccaccacacccggctaatttttgtatttttagtagagacagggtttcaccatgttggtcaggctggtctcgaactcctgacctcatgatccgcgcgcctcagcctcgcaaagtgctgggattacgggagtaagccaccatgcccggcctatcttctcatttcttaacattctttggaaatacagtgagcaaactagcatttgagtccctactatcctgatcataaagtgctttaaaatgtttttccatttaattcccactgactgggtgcgatggatcatgcttgtaatcgccgcactttgggagcctgaggcaggtggataacctaaactcaggagtttgagactagcctggccaacatggcgaaaccccctctgtacgccgggtgcggtggctcacgcttgtaatctagcactttgggaggccgaggcgggtggatcacgaggtcaggatcgagaccatcctggctaacacagtgaaaccccgtctctactaaaaatacaaaaaattagctgaccgtggtggcgggtgtctgtagtcccagctactcgggaggctgaggcaggagaatggcatgaacctgggaggcggagcttgcagtgagcccagatggtgccactgcactccagcctgggcgagagagcgagactctgtctcaaaaaaaaaaaaaaaaaaaagaaagaaaaagaaaaaaagaaagaaagaaaccctgtctgtactaaaaatacaaaaattagccaggcgtgtttgcgggcacctataataccaactactcaggaagctgaggcagaagaatcgcttgaacccaggtggcctggtcctgtagtcttagctacttgggaggatcgctcaatgctcaggaatttgaggttagagtgagctaagataccattgcactccagcctgggc

SEQ ID NO: 3 - DNA, 1833 bp, Homo sapiens
  source 1..1833: mol_type = genomic DNA; note = gene G6PT sequence -330 to +1502
ctgtgtgttcctacaacacctttcccagactgtgagcaacttgggaacaatgattatcaccttagtgttcggtgtgttttgtagaggagggcactcactaaaaactggtttgttgaatgcaaccacttaagaattggacatgccgtgcccagcccctaaggggcgctatgctcgttcgtccaaggagcagcgtgaccagcgggagggagtaagtcgacgtccccggcatgtggggcggggaactccactggccggcaagcgaagcagcaggcgaagaccacgcccccgccggccgcgcttgcgcagccttcgctagccccgccccgtcctattcgggctcccgcctctgttcaggtaggaggcggtatgcgggggaggggagtcacctgagggcggggatctcaggtcttggggagccattgttggcgaaccccaacttccgatacgacccggagctgttcctgcagttccagagacaattcatgggtcttggggccaccgaggcgctgtccctgaccaccagcacgagacccctttctatcgcgccagtcctgtggtctccgcacctctccagctcctgcacccccggcccccgtggttcccagccgcacagtagcgtgtcctgggtagcgtgaggacccacggggctgggcaggtgccacgagcccgccgcctcttcgccgcccgccgcctctcctcctctcccgcccgccgcctggccctcccctaccaggctgagcctctgggtgccagaagcgcggggcctccgggagaatacgtgcggtcgcccgctccgcgtgcgcctacgccttctgctccagttgctttcccaattgagcggaaaagccggggcatgttgccggggccctgggcgggacggttgtgccctgcagcccgaagcccgccggggcaccttcccgcccacgagctgcccagtccctctgcttgcggcccctgccaacgtcccacaggttggaaaccagaccctgcagcccccagctagcccgaaggaagcccagactgacgtcgcagggtaatggggacccagaggtagctctgtggggccccatcaggacctcagcccttagcggggctttctgtgtgacaccgtgggacctgaactggggaggtcccccttgcacagaattacaaggggcaactgtaggaaacaattaaatctattatgtgctaagtattgagagttccacggattgaggccctctctagaaggcttagaagtggggtagccggggtatgtgtctgactttgtgttttggtgcggtggtggtgagtgggtgtagaaactaatagtgccaagtgcagtgcctgtcaactagtaagttaataaaccttccgtgaatgaatgagaaggttctgtgactgagggagcagggctagagagggcatctctgctcatcccacccctgcctcccctgggctcaggcagacaaaagtaattaggttgaacctttaatcaaagctggtttcacaggcaagcagacatgatagaggaggtaaaggcagaaatcctggggacacagggtgctggcctggctcacaggcatgcctccttccgggacctcctccaccccctacagtttggcgctcagtaatctcttgttttcttgtctccctcaggacactgggtccccttggagcctccccaggcttaatgattgtccagaaggcggctataaagggagcctgggaggctgggtggaggagggagcagaaaaaacccaactcagcagatctgggaactgtgagagcggcaagcaggaactgtggtcagaggctgtgcgtcttggctggtagggcctgctcttttctacc

SEQ ID NO: 4 - DNA, 2301 bp, Homo sapiens
  source 1..2301: mol_type = genomic DNA; note = 2.3 kb of human G6PT sequence upstream of the translation initiation site
tatctcatctcctcctcctcccttccctgaccatcttctctggagtaaaagatctttgctctcaattttggaacacttacaattccttcagccactcttgttaggaagatataagggaagagagcgagagacaggaggcaactcaacggccaaacaggtttattcacaggaataagcctgcaaggagttccagtcaggagtctggctgagaccctgatcgcttacaagctgaggcttttataatatcgtttctattggggagaggtcgggaagtgctggctgattgggactgttggggactttgttaggctatgttaccttctgttgaagctatgggcagggttaacatttgctctgtttctgagaacacagtcatcattgttgtaaaatagcatcactactgttactcctcacaactcttaactgggatcagtgaaaaccattgttgttgagaactcttatagaatttctgagctcttatctaaatcatctgttgagtcttcacaatgatctaccaggctggtaggaaacgggagctcatagagattaaccaaggtcatacagttagaatgctgtgggtctaggactagaatttgatatttactccaggccccgtctctgtccccaacaatctaaccacaagttaaaattccaaattgtgggctgggcgcggtggctcacgcctgtaatcccagcactttgggaggccgaggtgagcggatcacgaggtcaggagatcgagaccatcctggcaaacacggtgaaaccccgtctctactaaaaatacaaaaaattacccgggcgtggtggtgggcgcccgtagtcccagctactcgggaggctgaggcaggagaatggtgtgaacccgggaggaggagcttgcagtgagccgaaatcgcgccactgcactccagcctgggcgactgagcgagactgtttcaaaaaaaaaaaaaaagaaaaaaagaaaatactgtgtgttcctacaacacctttcccagactgtgagcaacttgggaacaatgattatcaccttagtgttcggtgtgttttgtagaggagggcactcactaaaaactggtttgttgaatgcaaccacttaagaattggacatgccgtgcccagcccctaaggggcgctatgctcgttcgtccaaggagcagcgtgaccagcgggagggagtaagtcgacgtccccggcatgtggggcggggaactccactggccggcaagcgaagcagcaggcgaagaccacgcccccgccggccgcgcttgcgcagccttcgctagccccgccccgtcctattcgggctcccgcctctgttcaggtaggaggcggtatgcgggggaggggagtcacctgagggcggggatctcaggtcttggggagccattgttggcgaaccccaacttccgatacgacccggagctgttcctgcagttccagagacaattcatgggtcttggggccaccgaggcgctgtccctgaccaccagcacgagacccctttctatcgcgccagtcctgtggtctccgcacctctccagctcctgcacccccggcccccgtggttcccagccgcacagtagcgtgtcctgggtagcgtgaggacccacggggctgggcaggtgccacgagcccgccgcctcttcgccgcccgccgcctctcctcctctcccgcccgccgcctggccctcccctaccaggctgagcctctgggtgccagaagcgcggggcctccgggagaatacgtgcggtcgcccgctccgcgtgcgcctacgccttctgctccagttgctttcccaattgagcggaaaagccggggcatgttgccggggccctgggcgggacggttgtgccctgcagcccgaagcccgccggggcaccttcccgcccacgagctgcccagtccctctgcttgcggcccctgccaacgtcccacaggttggaaaccagaccctgcagcccccagctagcccgaaggaagcccagactgacgtcgcagggtaatggggacccagaggtagctctgtggggccccatcaggacctcagcccttagcggggctttctgtgtgacaccgtgggacctgaactggggaggtcccccttgcacagaattacaaggggcaactgtaggaaacaattaaatctattatgtgctaagtattgagagttccacggattgaggccctctctagaaggcttagaagtggggtagccggggtatgtgtctgactttgtgttttggtgcggtggtggtgagtgggtgtagaaactaatagtgccaagtgcagtgcctgtc

SEQ ID NO: 5 - DNA, 1290 bp, Homo sapiens
  source 1..1290: mol_type = genomic DNA; note = Wild type human G6PT (SLC37A4), hSLC37A4[NM_001164277.1
atggcagcccagggctatggctattatcgcactgtgatcttctcagccatgtttgggggctacagcctgtattacttcaatcgcaagaccttctcctttgtcatgccatcattggtggaagagatccctttggacaaggatgatttggggttcatcaccagcagccagtcggcagcttatgctatcagcaagtttgtcagtggggtgctgtctgaccagatgagtgctcgctggctcttctcttctgggctgctcctggttggcctggtcaacatattctttgcctggagctccacagtacctgtctttgctgccctctggttccttaatggcctggcccaggggctgggctggcccccatgtgggaaggtcctgcggaagtggtttgagccatctcagtttggcacttggtgggccatcctgtcaaccagcatgaacctggctggagggctgggccctatcctggcaaccatccttgcccagagctacagctggcgcagcacgctggccctatctggggcactgtgtgtggttgtctccttcctctgtctcctgctcatccacaatgaacctgctgatgttggactccgcaacctggaccccatgccctctgagggcaagaagggctccttgaaggaggagagcaccctgcaggagctgctgctgtccccttacctgtgggtgctctccactggttaccttgtggtgtttggagtaaagacctgctgtactgactggggccagttcttccttatccaggagaaaggacagtcagcccttgtaggtagctcctacatgagtgccctggaagttgggggccttgtaggcagcatcgcagctggctacctgtcagaccgggccatggcaaaggcgggactgtccaactacgggaaccctcgccatggcctgttgctgttcatgatggctggcatgacagtgtccatgtacctcttccgggtaacagtgaccagtgactcccccaagctctggatcctggtattgggagctgtatttggtttctcctcgtatggccccattgccctgtttggagtcatagccaacgagagtgcccctcccaacttgtgtggcacctcccacgccattgtgggactcatggccaatgtgggcggctttctggctgggctgcccttcagcaccattgccaagcactacagttggagcacagccttctgggtggctgaagtgatttgtgcggccagcacggctgccttcttcctcctacgaaacatccgcaccaagatgggccgagtgtccaagaaggctgagtga

SEQ ID NO: 6 - protein, 369 aa, Homo sapiens
  source 1..369: note = amino acid sequence of human G6PT
AISKFVSGVLSDQMSARWLFSSGLLLVGLVNIFFAWSSTVPVFAALWFLNGLAQGLGWPPCGKVLRKWFEPSQFGTWWAILSTSMNLAGGLGPILATILAQSYSWRSTLALSGALCVVVSFLCLLLIHNEPADVGLRNLDPMPSEGKKGSLKEESTLQELLLSPYLWVLSTGYLVVFGVKTCCTDWGQFFLIQEKGQSALVGSSYMSALEVGGLVGSIAAGYLSDRAMAKAGLSNYGNPRHGLLLFMMAGMTVSMYLFRVTVTSDSPKLWILVLGAVFGFSSYGPIALFGVIANESAPPNLCGTSHAIVGLMANVGGFLAGLPFSTIAKHYSWSTAFWVAEVICAASTAAFFLLRNIRTKMGRVSKKAE

SEQ ID NO: 7 - DNA, 20 bp, synthetic construct
  source 1..20: mol_type = other DNA; note = forward primer
gtgatcttcagcgccatgtt

SEQ ID NO: 8 - DNA, 20 bp, synthetic construct
  source 1..20: mol_type = other DNA; note = reverse primer
gaacttgctgatggcgtagg